(19) United States
(12) Patent Application Publication
(10) Pub. No.: US 2024/0355442 A1
(43) Pub. Date: Oct. 24, 2024

(54) Method For Producing Patient-Optimised Dosage Forms

(71) Applicant: DiHeSys Digital Health Systems GmbH, Schwäbisch-Gmünd (DE)

(72) Inventors: Markus Dachtler, Ulm (DE); Gerald Huber, Plüderhausen (DE)

(21) Appl. No.: 18/258,742

(22) PCT Filed: Dec. 7, 2021
(86) PCT No.: PCT/EP2021/084678

§ 371 Date: Oct. 12, 2023

(30) Foreign Application Priority Data
Dec. 21, 2020 (EP) 20212320.4

(51) Int. Cl. G16H 20/10; A61K 9/20; B33Y 10/00; B33Y 50/02; G16H 10/60

(52) U.S. Cl. CPC G16H 20/10; A61K 9/2095; B33Y 10/00; B33Y 50/02; G16H 10/60

(57) ABSTRACT

The present application relates to methods of personalized pharmaceutics. More specifically, the invention relates to methods for the production of pharmaceutical dosage forms, which are produced by 3D and/or 2D printing, whereby a wide variety of parameters relevant to the administration of the dosage form can be changed depending on the analysis of patient-and disease-specific data and can be adapted in the course of the patient's treatment. In this way, the quality of treatment for the patient can be improved in the future and significantly increased from the point of view of patient safety.

### FIELD OF THE INVENTION

The present application relates to methods of personalized pharmaceutics. More specifically, the invention relates to methods for the production of pharmaceutical dosage forms, which are produced by 3D and/or 2D printing, whereby a wide variety of parameters relevant to the administration of the dosage form can be changed depending on the analysis of patient-and disease-specific data and can be adapted in the course of the patient's treatment. In this way, the quality of treatment for the patient can be improved in the future and significantly increased from the point of view of patient safety.

### BACKGROUND OF THE INVENTION

With existing pharmaceutical manufacturing processes, the implementation of personalized medicine for each individual patient is either not feasible or can only be realized with a very high effort and inconvenience, as the preparations of individual active ingredients or, even more rarely, combinations of active ingredients, are only available in a few standardized doses and dosage forms. This applies above all to solid dosage forms, which are, however, the dosage forms usually best tolerated by patients (e.g. tablets, capsules, thin films, etc.).

The problem underlying the invention is therefore to provide a method which makes it possible to provide a dosage form of the required active substance(s) which is continuously optimized for each individual patient depending on his/her individual and disease-specific parameters in the course of a therapy with one or more active substances.

The above problem is solved by the embodiments of the present invention characterized in the present description and claims.

### SUMMARY OF THE INVENTION

In particular, the invention provides a method for producing patient-optimized pharmaceutical dosage forms comprising the steps of:
(1) Analyzing of individual and/or disease-related data of a patient suffering from a disease state, wherein, optionally, the patient may be exposed to an active ingredient for the treatment of the patient's disease state;
(1a) where appropriate, selecting one or more active ingredients to treat the patient's disease state, if the patient has not already been exposed to an active ingredient to treat the disease state; or
(1b) where appropriate, selecting one or more other active ingredients for the treatment of the patient's disease state, if the previous active ingredient(s) has disadvantages compared to the other active ingredient based on the analysis of step (1) for the treatment of the patient's disease state;
(2) determining at least one administration-relevant parameter of the previous active ingredient or of the active ingredient(s) selected according to step (1a) or (1b) for a pharmaceutical form from the analysis of the data according to step (1), where appropriate taking into account potential side effects and/or influences of further active ingredients for the treatment of the same or another disease of the patient to which the patient is exposed;
(3) printing a first dosage form containing the active ingredient(s) according to the at least one administration-relevant parameter determined in step (2) by means of 3D and/or 2D printing, wherein the at least one administration-relevant parameter is converted into one or more corresponding printing parameters for the 3D and/or the 2D printing;
(4) analyzing individual and disease-related data of the patient under administration of the first dosage form;
(5) adjusting the at least one administration-relevant parameter of the active substance or substances for a dosage form according to the analysis of the data according to step (4);
(6) printing a further dosage form containing the active ingredient(s) according to the at least one administration-relevant parameter adjusted in step (5) by means of 3D and/or 2D printing; and, optionally
(7) repeat steps (4) to (6).

The method according to the invention can be designed in such a way that the patient has not yet been treated with an active ingredient. This case is described in the optional step (1a), in which one or more active ingredients indicated for the specific disease are first selected.

According to the invention, the term “disease” or “disease state” of a patient includes, on the one hand, any pathological condition which is treatable with pharmaceutical dosage forms, wherein “treatable” according to the invention means that the course of the pathological condition is positively influenced under the respective medical circumstances. For example, in the case of serious diseases such as cancer and tumor diseases, this may be a statistically significant increase in the probability of survival over a period of time typically observed for the respective disease compared to the course of the disease in the absence of treatment. In other cases, “treatable” usually means that the disease condition at least does not worsen, preferably improves, ideally that the disease condition is cured. In still other conditions, such as transplants, the term “treatable” includes that the graft has a greater period of survival over the untreated state. According to the invention, the term “disease” or “pathological condition” also includes an existing risk of the patient developing a pathological condition in the future, so that the method according to the invention naturally also includes the production of dosage forms which serve the prevention and rehabilitation of pathological conditions, respectively.

The method according to the invention can be applied, as indicated in optional step (1b), for those cases in which the patient has been treated with one or more active agents (i.e. one or more “previous” active agent(s)), but due to any circumstances (e.g. intolerance, drug-drug interactions, unsatisfactory course of the disease under treatment with the previous active ingredient(s)), the previous active ingredient(s) is (are) replaced by another/other active ingredient(s) that is (are) also indicated for the respective disease.

In other embodiments, the method is used to optimize existing therapies with one or more active ingredient(s) without first selecting one or more active ingredient(s) after the data analysis in step (1), i.e. the method process is continued with step (2) after step (1).

In step (1) of the method according to the invention, individual and/or disease-related data (hereinafter also referred to as “individual parameters” or “disease-related parameters”) of the patient are analyzed. These are usually available in a medical record at a health care provider such as a treating physician or at a medical institution such as a hospital, a clinic and/or a health resort, preferably in digitized form. “Individual” data of a patient are personal parameters of the patient that are generally independent of the disease per se, but typically have or at least may have an influence on the disease per se or on its course. Individual data which are or can be analyzed in step (1) of the method according to the invention are preferably selected from age, developmental state, gender, genetic predispositions, height, weight, body surface area, body mass index, general physical condition, drug consumption (such as, for example, consumption of “soft” drugs such as, preferably, alcohol, nicotine, marijuana, etc.) and/or consumption of “hard” drugs such as cocaine, heroin, methadone, etc.), eating habits (such as fat, carbohydrate and protein content, timing of food intake, regularity of food intake) and drinking habits (such as preferably daily drinking amount), sleeping habits, physical activity and combinations of two or more thereof. “Disease-related data” of a patient refers to parameters that inform about the patient's disease state and are usually collected through diagnostic procedures. Disease-related data collected in this way, which are analyzed or can be analyzed in step (1), are preferably selected from blood pressure, heart rate, ECG findings, EEG findings, sonographic findings, CT findings, MRI findings, biopsy findings of diseased tissue, blood count, electrolyte blood levels, blood liver levels, nephrological blood and urine levels, blood lipid levels, blood glucose levels, vitamin metabolism data, metabolic interactions, medication schedule, side effect profiles, urine status, virological findings, bacteriological findings, fungal findings, parasitic findings, stage of disease, course of disease and combinations of two or more thereof.

Of course, the disease-related data depend on the disease to be treated with the dosage forms to be produced. These are described below in more detail, using some typical examples: diseases usually treated with oral dosage forms are, for example, diseases of the internal organs such as intestine, kidney, liver, pancreas, gall bladder etc. In the case of kidney, liver or pancreas transplantation, a very precise adjustment of the immunosuppressive medication, i.e. one or more immunosuppressants such as a calcineurin inhibitor (such as ciclosporin and/or tacrolimus and/or everolimus), a glucocorticoid (such as hydrocortisone and/or methylprednisolone), and/or an inosine monophosphate dehydrogenase inhibitor (such as a mycophenolic acid derivative or salt, e.g. mycophenolate mofetil or mycophenolate sodium essential). In this case, for example, individual data of the patient such as, in particular, weight, sex and/or age or body surface area, as well as data having or at least potentially having influence on parameters relevant to the administration (including amount of active ingredient, required coatings (e.g. enteric coating(s), etc.; see step (5)) of the immunosuppressant such as in particular whether it is a first or further transplantation, whether and if so which pre-existing and/or concomitant diseases of the patient which may have an influence on the dosage of the immunosuppressants, such as for example existing allergies, intolerances, potential interactions of the immunosuppressants to be administered, are analyzed. In another embodiment, if according to step (1b) the e.g. transplanted patient is already undergoing therapy with one or more immunosuppressants, a different immunosuppressant (e.g. instead of ciclosporin) is selected, e.g. due to one or more intolerances of the patient to the previously administered immunosuppressant(s) (e.g. instead of ciclosporin (previous active ingredient), tacrolimus (the other active ingredient) or several other immunosuppressants (e.g. instead of ciclosporin (previous active ingredient), tacrolimus (other active ingredient) and instead of mycophenolate mofetil (previous active ingredient), mycophenolate sodium (other active ingredient) should be administered. In yet another embodiment (i.e. without selection of one or more first active agents (step (1a)) and without selection of one or more other active agents (step (1b)), the result of the analysis according to step (1) is transferred to the next step (2) for selection of the administration-relevant parameter(s) of the immunosuppressant(s). In certain embodiments, at the beginning of an immunosuppressive therapy, i.e. methods including step (1a), a low dosage is initially determined in order to then prepare successively higher dosage forms of the immunosuppressive agent(s) in the further course of the method (steps (2) to (6)) according to the course of analysis of the corresponding individual and/or disease-related parameters (so-called uptitration). In yet other embodiments (e.g. in the case of cortiocoid dosage forms), a tapering of one or more active substances is realized by the method according to the invention in the case of an already existing therapy or in the further course of the therapy, in that dosage forms with successively lower doses of the immunosuppressant(s) (as in the example of corticoids here) are prepared in steps (2) and (3).

Of course, the up-and down-titrations described above in relation to immunosuppressants are also transferable to other active substances.

In another exemplary embodiment, dosage forms are produced for the treatment of rheumatological diseases. Individual patient parameters, as already described above, are analyzed. In addition to typical physical characteristics such as the presence and extent of joint swelling, the disease-specific parameters preferably include blood values such as inflammation parameters, in particular the so-called rheumatoid factor (see e.g. E. Feist, K. Egerer, G.-R. Burmester, Z. Rheumatol. 2007, 66:212-21) as well as antibodies against cyclic citrullinated peptides (ACPA). In addition to the aforementioned immunosuppressive agents, other active ingredients or classes of active ingredients such as tyrosine kinase inhibitors, e.g. erlotinib, are also used in the manufacturing process according to the invention.

Non-limiting further preferred indication areas and active ingredients or classes of active ingredients, respectively, connected therewith are e.g. oncology (example substances also tyrosine kinase inhibitors), neurological diseases such e.g., Parkinson's disease (active ingredients are e.g., dopamine antagonists such as ropinirole), hematology, i.e. hematological diseases such as anemias (exemplary active ingredients include iron preparations, vitamin B12 and folic acid) and myelodysplastic syndrome (MDS), and cardiovascular diseases (active ingredients are e.g., anticoagulants such as acetylsalicylic acid, vitamin K antagonists such as e.g., phenprocoumon and warfarin, thrombin inhibitors such a dabigatran, factor Xa inhibitors such as e.g., apixaban, for the treatment of stroke, ventricular fibrillation and for the prophylaxis of myocardial infarction, i.e. the risk of myocardial infarction). With respect to cardiovascular diseases high blood pressure is particularly to be mentioned, which often occurs together with or often represents the starting point for developing the mentioned diseases such as stroke and myocardial infarction (or being at risk of suffering stroke and/or myocardial infarction, respectively). Active ingredients useful in the invention in the context of high blood pressure are antihypertensives such as calcium antagonists, betablockers, ACE inhibitors, diuretics and AT1 blockers. Thus, it is beneficially possible according to the invention, depending on clinical parameters, in particular blood pressure, ECG, sonographic findings, preferably of the peripheric vessels, coronary vessels, lung vessels and/or brain vessels, as well as blood parameters such as levels of creatin kinase and/or troponin I, to adapt the medication with the method of the invention such as by selecting the respective anticoagulant(s) and/or the antihypertensive as well as respectively adapted combinations thereof und/or amount(s) of active ingredient(s).

An adaptation of administration-relevant parameters for the additive printing of dosage forms with optimized dose-effect or dose-side-effect relationship by the method according to the invention can advantageously also be used in pediatrics and geriatrics. In the case of younger patients, in particular children, the method according to the invention can be used to adapt the size of the printed dosage form to the individual patient, in particular since dosage forms to be administered orally can only be taken with difficulty, starting from an individually different size, of course. The method according to the invention also makes it possible to realize dosage forms for pediatric patients with a particularly fine adjustment of the dose-relevant parameters, since it is particularly important in children and adolescents to precisely adjust the dosage to the age and developmental state of the patient, to reduce side effects and to reduce cross-reactions with other active ingredients. In geriatrics, in turn, the process according to the invention is used to print patient-optimized dosage forms which are also adapted to the special needs of elderly patients, in particular those aged 70 years or more, preferably 75 years or more, particularly preferably 80 years or more. This concerns, inter alia, the printing volume, since elderly patients may have difficulty grasping small-volume dosage forms. Also, the method according to the invention can be used to efficiently switch an elderly patient from a classical dosage form such as a tablet to a suckable dosage form, as geriatric patients often suffer from swallowing difficulties. Both pediatric and geriatric patients will benefit from the method according to the invention, inter alia, in the respect that polypharmaceutical dosage forms, i.e. dosage forms with several active ingredients, can be produced and individually adapted in the case of a required administration of several active ingredients per day or per time of intake. In general, all patients benefit from the optimization of individual dosage forms with multiple active ingredients in that, e.g. if the patient is to take an additional active ingredient, an existing dosage form, e.g. with one or more other active ingredient(s) already administered, can be modified in such a way that the new active ingredient (it can of course also be several new active ingredients) is administered together with the other active ingredient(s), optionally, through defined areas within the dosage form, each of which containing an active ingredient and which are separated from the other areas with the other active ingredient(s), for example by barrier layers, and printed into a new, patient-individually dosed dosage form (step (3) and/or step(s) (6) of the method according to the invention).

Other diseases within the scope of the present invention are, for example, those of the cardiovascular system such as high blood pressure, arteriosclerosis, cardiac arrhythmia, angina pectoris, heart attack, stroke, etc.

It is apparent to a person skilled in the art that the dosage forms which can be produced by the method according to the invention are not limited in any way with respect to the active ingredient and the parameters related to the treatment of the respective disease. A person skilled in the art can select the disease-related parameters to be considered for a specific indication in each case on the basis of his expertise and take them into account for the production of the respective dosage form.

In step (2) of the method according to the invention, at least one administration-relevant parameter of the (previous) active substance or of the active substance(s) selected according to step (1a) or (1b) is selected for a dosage form from the analysis of the data according to step (1), if necessary taking into account potential side effects and/or influences of further active substances for the treatment of the same or another disease of the patient to which the patient is exposed.

An “administration-related parameter” of a dosage form of the active ingredient(s) (either of one or more active ingredients already administered to the patient (i.e. without step (1a) or step (1b)) or of one or more new active ingredients for the condition (i.e. step (1a) is performed) or of one or more other active ingredients (i.e. step (1b)) is any parameter of a dosage form that affects the drug dose, drug concentration, drug release, route of administration, administrability, tolerability and/or, stability (to physical and/or chemical parameters) and efficacy of a dosage form of the active ingredient(s). Typical and preferred administration-relevant parameters are, for example, amount of active ingredient(s) per unit dose of the dosage form, release kinetics of the active ingredient(s) from the dosage form at the site of administration and/or along the route of the dosage form in the patient (pharmacokinetics and pharmacodynamics), concentration of the active ingredient(s) in the dosage form, concentration distribution of the active ingredients or ingredients in the dosage form, size of the dosage form, geometric shape of the dosage form, coating parameters of the dosage form, surface structure of the dosage form, internal structure of the dosage form, distribution of the active ingredient or ingredients in the dosage form, and combinations of two or more of these parameters.

In the case of the method with step (1a) (new active ingredient(s), previously no active ingredient), all the administration-relevant parameters required according to the analysis of the data for the dosage form and adapted (at the actual time point), thus prima facie optimal, are usually determined. This will usually also be the case in the embodiment of the method according to the invention in which step (1b) is carried out (selection of one or more active ingredients other than the previous active ingredient(s)). The method according to the invention is not limited to optimizing dosage forms of individual patients. Rather, according to the invention, it is also envisaged to record the parameters of optimized dosage forms of several patients, preferably a plurality of patients, such as for example to store them in a database on one computer or several or a plurality of connected computers, and to make them usable for the provision of optimized dosage forms for other patients who have similar individual and/or disease-related parameters as patients whose optimized dosage forms are already known, i.e. are preferably stored in the database. Such embodiments can thus make the therapy of further patients more successful more quickly. Furthermore, it is also envisaged according to the invention to evaluate and train using existing and thus further recorded parameters of dosage forms with artificial intelligence methods and devices, such as, for example, deep learning methods and corresponding devices, in order to further improve the optimization method according to the invention. In such embodiments, therefore, one or more (initial) active ingredient(s) is preferably selected and parameterized in step (1a) and subsequently in step (2), and a corresponding first dosage form is printed in step (3), which is provided on the basis of the dosage forms known in the existing database from cases of the same and/or similar individual and/or disease-related parameters, after which the further process steps follow. In the embodiment of the method according to the invention, in which a dosage form is prepared which already contains one or more with which the patient has already been treated, but the dosage form is to be adapted to the individual and disease-related parameters by the method according to the invention (i.e. the optional steps (1a) or (1b) are not carried out), one or more parameters relevant to the administration are selected in step (2). In certain embodiments of the method, this may be only one parameter, such as, compared to the existing medication, a change in the dose of the active ingredient, a change in the release kinetics or a change in the size of the dosage form, etc. Of course, any combination of administration-relevant parameters can also be selected in step (2), as required on the basis of the analysis performed in step (1), in order to adapt the dosage form to the analyzed individual and disease-related parameters of the patient. In this embodiment (pre-) optimized dosage forms can also be created in accordance with the steps described above using available parameters from cases that are already the same or similar, preferably using artificial intelligence methods and devices such as deep learning methods and corresponding devices, and then further adapted if necessary.

When determining the parameters relevant to the administration, potential side effects and/or influences of further active substances for the treatment of the same or another disease of the patient to which the patient is exposed are taken into account, if necessary. This is a particular advantage of the method according to the invention compared to standardized dosage forms, as it not only takes into account the (variable) individual and disease-specific parameters of the patient when preparing the dosage form of the active ingredient or the active ingredients, but also ensures that the active ingredients printed into the dosage form to be prepared have at least an improved side effect or influence profile with respect to these other active ingredients, as well as with respect to other active ingredients administered to the patient. Ideally, the optimal dose for maximum therapeutic success with a minimum side effect profile should be applied to the patient.

After determining the administration-relevant parameter(s) according to step (2), these are made available to a 3D and/or 2D printing process, generally an additive printing process, which, if not all the required (new) administration-relevant parameters have been determined in step (2), takes over the further parameters from otherwise usual or already existing parameter values and creates the dosage form with the aid of a 2D or 3D printing process that is in principle freely selectable.

Printing processes useful for generating the dosage form in step (3) and/or step(s) (6) of the process according to the invention include, for example, extrusion processes such as filament fusion fabrication (FFF) or fused layer modelling (FLM), jetting processes such as voxel printing (also called direct jetting) or binder jetting, and spot printing processes.

### DETAILED DESCRIPTION OF THE INVENTION

An extrusion printing process preferred according to the invention is described in WO 2020/240030 A1 and comprises the steps of
providing a pharmaceutical printing device designed for FFF or FLM 3D printing,
providing starting objects adapted for FLM or FFF 3D printing, said starting objects forming at least a first and a second group of starting objects, wherein the composition of the starting objects of the first group is different from the composition of the starting objects of the second group or at least one parameter according to the invention as set out above is different between the starting objects of said groups, and at least one group of said starting objects contains at least one active agent selected from the group consisting of pharmaceutical, nutraceutical and food supplement active agents; and
printing the starting objects of the at least first and second groups in the form of filaments and inks, respectively, with the printing device until the dosage form is created.

The preferred extrusion process can in principle be carried out with known components of FFF 3D printing or FLM 3D printing, whereby in relation to FFF processes reference can be made, for example, to the relevant disclosure in WO2016/038356 A1.

A “starting object” in the sense of the preferred extrusion printing process are materials suitable for FFF 3D printing or FLM 3D printing, which can be processed in a basically known manner by corresponding printing devices and printed to form a 3D object, wherein in said processes the starting object or synonymously the starting material comprises a base composition printable in filament form. Typically, in the extrusion printing process, the starting objects are converted to a flowable state, typically by heating by a heating device located in print head of the printing device, and then applied, typically in layers, by the print head, typically by extrusion, in filament form on a build platform. The method according to the invention can also be designed in such a way that the starting objects are printed in filament form on an already existing object present on the build platform of the printing device.

The starting material or starting objects, respectively, for the preferred FFF 3D process are filaments that typically have the shapes given as examples in relation to the filament structures.

Starting material or starting objects, respectively, for a FLM process are typically granules, pellets, powder and/or flakes.

In one embodiment of the preferred extrusion process, the starting objects of one group comprise at least a first active ingredient or a first active ingredient composition and the starting objects of the or another group comprise a second active ingredient or a second active ingredient composition, wherein the second active ingredient is different from the first active ingredient or the second active ingredient composition is different from the first active ingredient composition.

In another embodiment of the preferred extrusion process, the starting objects of the other or another group contain the same active ingredient, wherein the concentration of the active ingredient in the starting objects of one group is different from the concentration of the active ingredient in the starting objects of the other group or groups. In other words, in this embodiment, starting objects or materials are printed which form at least two groups, both groups or (in the case of more than two groups) at least two groups containing the same active ingredient, but each at a different concentration. It is provided in a further preferred embodiment that more than two, preferably 3, 4, 6, 7 or 8, groups of starting objects are provided and printed in the form of filaments, each group containing an active ingredient which is different from the respective other active ingredients of the other groups of starting objects. In another embodiment, more than one active ingredient, e.g. 2, 3, or 4 active ingredients, with 2 active ingredients being particularly preferred, are present in one or more groups of starting objects.

At least one group of starting objects may also be present, i.e. printed, which does not contain any active ingredient, so that solidified filament structures are present in the prepared dosage form which do not contain any active ingredient.

In another embodiment of the preferred extrusion process, the groups of starting objects or materials may be such that the printed filaments differ in drug release characteristics.

It is of course also possible to combine the above-mentioned different characteristics of the starting objects or the printed filaments in the preferred extrusion process.

In a further embodiment of the preferred extrusion process, each group of starting objects having the same composition (or having at least one same essential parameter of the invention as set out above) has a detectable distinguishing feature that is different from the other group(s) of starting objects, so that the printed filaments having different compositions (or different parameters, respectively) can be differentiated. As stated above, preferred distinguishing features include, for example, filament diameter, length of filament, visible dyes, fluorescent dyes, surface textures, shape, gloss, porosity, roughness and absorption or reflection properties with respect to electromagnetic radiation.

Preferably, the extrusion process is carried out in such a way that one or more layer(s) of the first group of starting objects is/are printed in the form of filaments and subsequently one or more layer(s) of the second group of starting objects is/are printed in the form of filaments and, if necessary, one or more layer(s) of the further groups of starting objects is/are printed in the form of filaments. The respective groups of starting objects can also be printed alternately in layers. This can also be done by means of layers without active substance, e.g. to optimize the chemical stability of the starting materials.

In another preferred embodiment of the preferred extrusion process, the groups of starting objects are printed such that the created dosage form, as described above, has at least one partial volume that does not contain printed filaments. This may be done in a manner known to those skilled in the art, in one embodiment by forming a cavity containing air or an inert gas (such as nitrogen). In another embodiment, the filaments may also be printed around a (solid or semi-solid) composition containing, for example, an identical or different active ingredient (compared to the active ingredient(s) in the printed filaments). In another preferred embodiment, the filaments are printed (e.g. by cross-layering in such a way as to create voids in the construction of the dosage form) so as to form several voids, preferably a plurality of voids, which are preferably in communication with the environment so as to form a sponge-like or porous, preferably highly porous, dosage form.

With respect to further preferred embodiments and details of the preferred extrusion process and printed dosage forms printed therewith, reference is made to WO 2020/240030 A1, the entire disclosure content of which is hereby incorporated by reference in the present description.

In another embodiment, the printing process is designed in such a way that instead of printing filaments, inks are printed that harden after application either by cooling or chemically or physically. For such a process, the steps mentioned above for the FFF process apply accordingly.

A preferred jetting process for creating a dosage form in step (3) and/or step(s) (6) of the method according to the invention is described in WO 2020/240028 A1, and comprises the steps:
(i) establishing a two-or three-dimensional representation of the object to be produced using predefined volume increments;
(ii) printing a predefined volume increment on a formation device or on an object placed on the formation device;
(iii) printing a further volume increment such that the volume increments at least partially contact or overlap; and
(iv) repeating steps (ii) and (iii) until the object is created;
wherein at least one of the volume increments contains at least one pharmaceutically active ingredient to be administered and the volume increments comprise a base composition or base substance which is flowable at a printing temperature compatible with the at least one active ingredient and which solidifies after printing of the respective volume increment and/or the volume increments are surface-adhered to one another.

In the sense of the preferred jetting process preferred according to the invention, the dosage form to be produced is a three-dimensional object, which according to the invention is to be understood in such a way that in the real world any object produced by two-or three-dimensional printing processes, in this case pharmaceutical dosage forms, extends in three spatial directions. Provided that in the jetting process preferred according to the invention only one layer of at least partially contacting volume increments is printed on the formation device or on an object already present on formation device, the jetting process preferred according to the invention can also be described as a 2D printing process. If the volume increments are applied, for example, as droplets which, for example, by subsequent moisture removal, for example by drying, macroscopically appear as two-dimensionally expanded units, these are, however, microscopically three-dimensional structures, so that, according to the invention, the dosage form also has a three-dimensional expansion in such embodiments according to the invention.

According to a preferred embodiment of the jetting process, the dosage form is built up layer by layer, i.e. in steps (ii) and (iii) the volume increments are printed layer by layer. Preferably, the volume increments are printed row by row or column by column.

The preferred jetting process according to the invention is characterised in particular by the high flexibility of the composition and the possible multitude of options for the formation of the semi-solid or solid dosage form produced. Thus, in preferred embodiments of the jetting process, different volume increments may have different active ingredients and/or different amounts of active ingredients and/or different base compositions or base substances. Furthermore, the shape and/or volume of the volume increments (hereinafter also referred to as “voxels”) may be the same or different.

In the preferred jetting process, it is of course also envisaged that the dosage form is constructed entirely from volume increments, all of which contain a single, identical active ingredient, it also being envisaged that each volume increment may contain the same amount of active ingredient or the same concentration of active ingredient.

The volume increments are essentially freely definable and can, for example, take the form of drops, spheres, points, cylinders, cubes, cuboids or other shapes. The geometric shapes mentioned (spheres, cylinders, cubes, cuboids) are to be understood according to the invention as meaning that the voxels essentially assume this shape, preferably when they have solidified after printing, so that voxel shapes which are also preferred according to the invention are also more generally pellets, which are preferably approximated to a spherical shape or a cylinder, and granulates. Preferred voxel shapes are thus in particular drop-shaped, pellet-shaped, cylindrical and granulate-shaped voxels. As already detailed above, the shape (e.g. the examples mentioned above) and the size of the volume of the volume increments can be freely combined essentially independently of each other.

Certain preferred embodiments of the method according to the invention make use of the fact that the volumes of the printed volume increments can in principle be freely selected in principle, the volume of a pharmaceutical dosage form shrinks from the outside to the inside during its degradation towards or at, respectively, the release site. This causes the amount of active ingredient released per unit time to decrease. In order to achieve a release of active ingredient that is as uniform as possible in this respect over the course of the degradation of the dosage form, it is provided according to the invention to print the volume increments in such a way that the volume of the printed volume increments increases from the outside inwards. According to the invention, this is realized by printing corresponding layers of volume increments, whereby the volume of the volume increments increases from layer to layer or from a group of layers of the same volume to further layers of the same volume from the outside inwards.

As already mentioned, different active pharmaceutical ingredients (APIs) can be contained in the dosage form by means of the preferred jetting process. In addition, several (i.e. two or more) active ingredients can be contained in the volume increments. Of course, volume increments may also be printed such that each volume increment contains one API, but different APIs (two or more) are present in separate volume increments. Volume increments can also be printed that contain different concentrations (i.e. amount of API per volume increment) of an API. In a related embodiment, the process can be designed such that active ingredient-containing volume increments are constructed and printed such that at least a first group of contacting or overlapping active ingredient-containing volume increments contains an equal amount of active ingredient and at least a second group of contacting volume increments contains an amount of active ingredient that is different from the amount of active ingredient of the first group. Thus, concentration gradients can be established in a dosage form prepared by the jetting method. This embodiment of the invention is also used in preferred variants of the invention to provide a uniform release of active ingredient, as described above for increasing the volumes of the volume increments in the dosage form from the outside to the inside. Thereby, to provide a release of the active ingredient(s) as uniform as possible, the volume increments are preferably printed in such a way that the concentration of active ingredient preferably increases in the volume increments from the outside to the inside.

In further embodiments, dosage forms can be provided in which the ratio of active ingredient release per unit of time is controlled by the surface area of the printed volume body (i.e. the printed dosage form) that is accessible to a surrounding medium. Thus, for example, for a zero-order release, a doubling of the active substance release will be realized by doubling the surface area (accessible to the surrounding medium), at least in a first approximation. In preferred embodiments, honeycomb structures can be printed, for example, which macroscopically have the shape of a usual dosage form (e.g. tablet), but which have a surface area many times larger with approximately the same size.

In the production of dosage forms according to the preferred jetting process, groups of volume increments with different active pharmaceutical ingredients can be formed. At least one first group of active ingredient-containing volume increments may be present, which contains a first active pharmaceutical ingredient, and at least one second group of active ingredient-containing volume increments may be present, which contains a second active pharmaceutical ingredient different from the first active ingredient. The different groups of volume increments may be printed in such a way that they are grouped together within the dosage form. This means that the volume increments of the first and/or the second group (and possibly of each further group if more than two active ingredients are to be present in the object to be printed) are printed in such a way that the volume increments of the respective group contact each other.

In further embodiments of the preferred jetting process, it is also provided that active substance-containing volume increments are printed in such a way that they form one or more groups within the object which are at least partially, in other embodiments also completely, surrounded by non-active ingredient-containing volume increments which separate or shield the active ingredient-containing volume increments from the external environment. so that, for example, a dosage form with an active ingredient-containing core or, at least, an inner group of interconnected active ingredient-containing volume increments (or several inner groups of adjacent volume increments with the same or different active ingredients or the same or different amounts of active ingredient) is produced, around which non-active ingredient-containing volume increments are arranged. The “external environment” can be the environment surrounding the object. The term “external environment” around a core region or an inner group of volume increments directly connected to one another is also understood to mean another region within the printed dosage form, i.e. volume increments not containing active ingredient can at least partially, and possibly completely, separate groups of volume increments containing active ingredient from other individual or groups of volume increments in the printed dosage form, or even completely surrounded by other individual or groups of volume increments containing, for example, another (or several other) active ingredient(s), in order to form separating layers or separating areas between the differently equipped volume increments.

In preferred embodiments, such arrangements can be used to spatially isolate the individual active ingredient-containing volume increments, e.g. in order to avoid chemical instabilities of the individual active ingredients and/or to separate different active ingredients that are chemically incompatible with each other (e.g. because they react with each other or otherwise impair their structure and/or efficacy).

In other embodiments of the aforementioned type, drug-abuse detergent tablets or capsules can also be provided, for example, which prevent an active ingredient (for example, opioids or active ingredients with addictive potential) from being extracted from a dosage form, for example, by crushing or in some other way, and from being misused. Thus, in preferred embodiments of the invention, groups or layers of volume increments are printed with the active ingredient(s) (e.g., the aforementioned potentially misusable substances), which are surrounded by groups or layers of volume increments containing a substance which cancels the effect of the active ingredient(s), degrades the active ingredient(s), or otherwise at least limits, and particularly preferably prevents, the potential misuse of the active ingredient(s). One or more groups or one or more layers of volume increments may also be provided between the groups (or layers) of active ingredient(s) and abuse-preventing substance(s), which contain no active ingredient and no abuse-preventing substance (in preferred embodiments, these volume increments will only contain the base building substance used) and separate the active ingredient-containing volume increments from the volume increments containing the abuse-preventing substance(s).

Furthermore, according to the invention, such embodiments with active ingredient-containing volume increments at least partially surrounded by non-active ingredient-containing volume increments can be used, for example, for the production of slow-release embodiments such as sustained-release tablets or capsules or enteric-coated tablets or capsules.

The preferred jetting process in the context of the present invention can thus be used to produce objects, in particular pharmaceutical dosage forms, which release the API(s) at a selected site or a selected area of the desired application (so-called “drug targeting”), i.e. preferably used for release control of the drug(s) from the printed dosage form. Such embodiments are thus used to deliver the drug or drugs to the optimal site of action or target, for example (and preferably) following oral administration. Thus, in certain embodiments of the invention, it is envisaged that the volume increments are printed in such a way that a core region of volume increments of a pharmaceutical dosage form according to the invention contains one or more desired active ingredient(s) and around this core region (or around this core volume) one or more layers of volume increments are arranged which, for example, are degraded in the intestine in a pH-dependent manner. These are degraded or dissolved, respectively, in the intestine in a pH-dependent manner, so that the core area is only exposed to the surrounding environment through the pH-dependent degradation of the outer layer(s) in the preselected area of the intestine and releases the active substance(s) there. This is mostly achieved by polymers (such as shellac, copolymers of methacrylic acid and methacryl methacrylate, modified celluloses, etc.) and/or polyvinyl alcohol derivatives, which are well known in the field and whose pH-dependent degradation or pH-dependent solubility can be very finely adjusted. pH-dependent solubility can be very finely controlled, so that, according to the invention, a pH-dependent exposure of the active substance-containing core region of the dosage form can be provided for each section of the intestine, in particular the small intestine (duodenum, jejunum and ileum). The provision of a targeted release at a specific site of action or target site, such as the intestine or a selected intestinal section, is not limited to pH-dependent degradable or pH-dependent soluble layers of volume increments with corresponding pH-dependent degradable or pH-dependent soluble polymers contained in such build-up substances. Other mechanisms can also be implemented alternatively or additionally. Thus, according to the invention, the volume increments can be printed in such a way that one or more layers of volume increments are formed, for example directly on a core region containing active substance or on one or more intermediate layer(s) which may be present and whose build-up substance contains or consists of a bacterially degradable component. Bacterially degradable polymers known to a skilled person, such as starches or celluloses, are suitable for this purpose. Such layers preferably serve to release active substances in the colon. In preferred embodiments, the aforementioned layers, e.g. pH-degraded layers (one or more) and bacterially degradable layers can be combined. In this way, it is also possible to print, for example, dosage forms for pharmaceutical active ingredient combinations, in which volume increments with a first active ingredient are provided in a core region, which is surrounded by one or more layers of volume increments which contain (or consist of) bacterially degradable substances in the build-up substance. This is followed by one or more layers of volume increments containing a second active ingredient, which is followed by one or more layers containing (or consisting of) one or more pH-degradable polymer(s) in the build-up substance. Alternatively, of course, in such an embodiment with multiple drug targeting layers, only the core region may contain one or more active ingredients.

Since the preferred jetting process can also be carried out under sterile conditions, the printing process according to the invention can also be used for the provision of implants and/or injections or active ingredient depots that release active substances.

Furthermore, the fact that very different materials (active ingredients and base compositions or substances) can be used for the volume increments to be printed also contributes to the increased flexibility of the preferred jetting process.

Bonding between the individual applied volume increments may be accomplished in a variety of ways. For example, in one embodiment, when a meltable material is used, the bonding between such voxels may be accomplished by solidification after application to the support structure, which may be accomplished by various mechanisms such as simple cooling and/or chemically by known substances. In another embodiment, a suitable binder may be added to the voxel material, e.g. a dispersion or a solution, which after application of the voxel causes it to harden, wherein the hardening by the binder may be effected, for example, by heat, which may be supplied by a suitable heat source in the printing device, such as a light source, preferably a laser device. Curing by the binder may also be effected chemically by appropriate starter molecules and/or light of a suitable wavelength, the latter again preferably being emitted by means of a laser device. In a further embodiment, the fluid of the volume increment may contain one or more starting compounds, typically monomers, of one or more polymers and, after application of the voxel, polymerization is initiated by suitable means such as, again, light, heat or other polymerization initiators, which cures and bonds the applied voxel to adjacent voxels.

With respect to further preferred embodiments and details of the preferred jetting process and dosage forms printed therewith, reference is made to WO 2020/240028 A1, the entire disclosure content of which is hereby incorporated by reference in the present description.

Suitable carrier materials flowable at the printing temperature, in which the active pharmaceutical ingredient(s) is/are present, are carriers such as low-melting waxes and polymers generally usable for hot melt extrusion (HME) in both extrusion and jetting processes. In addition to the low melting point carrier, the HME mixture or, more generally, the volume increment mixture may contain other process agents and excipients such as binders, fillers, plasticizers, antioxidants, fragrances, sweeteners or the like. Suitable HME carriers and plasticizers and are disclosed, for example, in Crowley et al. (2007) Drug Development and Industrial Pharmacy, 33,909-926 (carriers: pages 917 to 919, in particular Table 1; plasticizers: pages 917 and 920, in particular Table 2), reference being made expressis verbis to the said passages in the present description.

A spot printing process suitable according to the invention in step (3) and/or step(s) (6) comprises the following steps:
(a) providing a printer capable of at least 3D printing the solid dosage form, the printer having
a build-up platform on which the dosage form is printed,
a print head designed for applying an array of spots of a build-up substance containing at least one pharmaceutically active ingredient for the dosage form on the build-up platform, wherein the build-up substance is flowable in the printed state, preferably by heating, and becomes at least semi-solid by solidification, preferably cooling,
(b) applying an array of spots of the build-up substance on the build-up platform, wherein the spots overlap or may or may not contact each other; or
(c) at least semi-solidifying, preferably solidifying, the spots of the build-up substance applied in step (b);
(d) applying a further array of spots to the previous array of spots such that the spots of the further array at least partially overlap with the spots of the previous array; and
(e) repeating steps (b) to (d) until the dosage form is formed.

A “spot” in the sense of the above printing process is an essentially round, inherently three-dimensional structure that results from the impingement of a volume unit of the build-up substance that is ejected from a print head of the printing device in liquid, at least flowable form, usually in the form of a drop, (approximate) rotational ellipsoid or (approximate) sphere, and is deposited on the build-up platform (in step (b)) or, at least partially, on previously deposited spots (in step (c) or steps (c)).

As mentioned above, dosage forms can be prepared by means of the invention, wherein build-up substances containing at least one pharmaceutically active ingredient are used. In preferred embodiments, synergistically acting combinations of 2 or more pharmaceutically active ingredients are provided, which may be present in a single build-up substance. In another embodiment, different active pharmaceutical ingredients may be present in different build-up substances.

The active ingredients in the preferred spot printing process may be present in groups of build-up substances that form the applied spots. Thus, according to the invention, an active ingredient may be present in a build-up substance from which a first group of spots is formed and one (or more) other active ingredient(s) may be present in another group of spots (i.e. at least two build-up substances are present which contain the respective active ingredient(s)). preferably, the different active ingredients are contained in different build-up substances. It is possible that, with the exception of the active ingredient(s), the respective composition substances can otherwise be the same or different, e.g. in order to provide properties tailored to the respective active ingredient, such as pH, solubility, consistency, ionic environment, particle size, color, viscosity, dissolution rate, temperature, isotonicity, etc., depending on the respective active ingredient. In certain embodiments of the invention, combinations of 2 or more pharmaceutically active ingredients are provided, in which, for example, a pharmaceutically active ingredient intended for a specific indication is contained in a composition and a further pharmaceutically active ingredient is present in the same or another composition, which, for example, at least reduces, at best suppresses, a side effect potentially caused by the first active ingredient, which represents a particularly preferred embodiment of the preferred spot printing process.

In preferred embodiments of the preferred spot printing process in the context of the invention, spots with the same active ingredient or with the same active ingredient combination or with the same active ingredient concentration are located in a common section of the dosage form, such that the spots of the same group are at least partially adjacent to each other on at least one side. Preferably, therefore, the spots with the same active ingredient or with the same active ingredient combination or with the same active ingredient concentration each form at least one common section such as (for example) at least one common layer or at least one contiguous part of at least one layer, it being possible for these to be oriented horizontally or vertically with respect to the longest dimension of the dosage form. In other embodiments, spots each having the same active ingredient, the same active ingredient combination or the same active ingredient concentration may also be combined in several sections (i.e., for example, 2 or more layers and/or partial layers). Such sections may also have different active ingredient release properties such as different pH conditions, solubility, gastric juice resistance, other solubility behavior (e.g. in which spots of certain sections or of a certain section contain a burst-release substance, wherein a method for forming a burst-release embodiment is preferably such that the spots of the burst-release sections are applied in such a way that the burst-release portions surround the spot portions without burst-release substance in the build-up substance, i.e. at least one, preferably several layers of spots with burst-release substance(s) in the build-up substance(s) in each dimension of the dosage form surround the portions of the dosage form without burst-release substance).

Preferably, the printer provided for the preferred spot printing method in the context of the invention comprises at least one print head which is in communication with a reservoir with the build-up substance, so that the at least one print head is capable of withdrawing a quantity of the build-up substance for applying the build-up substance in steps (b) to (e). The reservoir may be configured in different ways depending on the type and consistency of the build-up substance. For example, in the case of liquid build-up substances, the reservoir can be a liquid container that is connected to the print head via a line for the build-up substance through which it is transported, usually pumped, to the print head. In another embodiment, the build-up substance may be present in the reservoir in solid, liquid or semi-solid form, for example as powder or granules, with a transport mechanism feeding the solid or semi-solid build-up substance to the printhead. In this embodiment, the print head typically has a heating or melting device which converts the build-up substance into an at least flowable, in preferred embodiments liquid form, which is then dispensed, i.e. printed, from the print head by a usually present dispensing device as a volume unit forming a spot on the build-up platform.

In another embodiment of the preferred spot printing process, the build-up substance can also be in the form of a solid or at least semi-solid filament, whereby the filament is present, for example, in a feed passage or a feed tube, which form the reservoir. These reservoir forms can be of different shapes, whereby linear embodiments are usually provided in the case of completely solid filament build-up substances. Preferred filament build-up substances are usually elongated, cylindrical structures, which are typically more or less elastic and can therefore also be accommodated in curved, such as spiral-shaped, reservoir coils and can be fed to the print head, for example, by a pushing or pressing mechanism. If the elasticity is not sufficient to feed the filament spirally in reservoir spools, then the filament can also be fed in short, straight filament rods from a reservoir magazine.

In the case of liquid build-up substances, the print head may comprise piezoelectrically driven means for dispensing the volume unit so that the build-up substance is dispensed as in an inkjet printer. Such an embodiment may also be configured as a 2D printing process, as detailed below. In a 2D printing process, a liquid (examples are given below) is typically deposited via known techniques such as piezoelectric dispensing devices, e.g., on at least a portion of the surface of a dosage form created by the above steps in 3D printing, e.g., in an additional step (vi), wherein the liquid is typically dried or otherwise fixed (e.g., chemically, physically, and/or by exposure to light, which is usually effected by a laser device) on the at least one portion of the surface. Of course, it is also possible to apply one or more 2D printed layers within a dosage form and to proceed with the 3D printing steps after applying a 2D layer (which may of course be followed by a final 2D printed layer).

In another preferred embodiment, the printer has more than one printhead, with 2 to 10 printheads being particularly preferred. Embodiments of the invention with multiple print heads may serve different functions: in one embodiment, it is envisaged that more than one printhead is used to print on a unit of the dosage form, e.g. to print different build-up substances with different properties as outlined above. 3D and 2D print heads may also be provided in an embodiment with multiple printers, and it is also possible, as explained below, that print heads usable according to the invention are designed for both 3D printing and 2D printing. It is further envisaged according to the invention to use more than one print head to print multiple dosage forms simultaneously. According to the invention, it is of course also possible to print several different dosage forms simultaneously, i.e. sets of print heads are formed, so to speak, or at least addressed as sets, which print simultaneously with different composition substances on several dosage forms, whereby in turn the dosage forms printed simultaneously may be identical or different in their composition. The number of print heads can therefore also significantly exceed 10 print heads in order to be able to increase the number of printed dosage forms, accordingly. In the embodiment of the method with more than one print head, it is further preferred that each of the print heads is in communication with a reservoir with the build-up substance, so that the respective print head is capable of withdrawing a quantity of the build-up substance for application of the build-up substance in steps (ii) to (v). The above reservoir embodiments may be the same or different for the print heads independently of each other.

The spots are preferably created according to the invention by applying a volume unit of the build-up substance, whereby a volume unit preferably has a volume of 20 pl to 30 μl. If necessary, several volume units can of course be applied in succession.

According to another preferred embodiment of the spot printing process, the print head or, in the case of multiple print heads, at least one of the print heads is adapted for both 2D printing and 3D printing. In another embodiment of the invention, the apparatus comprises at least one or more 3D print heads and, if required, a 2D print head. 3D print heads are designed for applying semi-solid and molten build-up substances, while 2D print heads are designed for applying build-up substances that are already liquid without heating in the print head, such as inks or active ingredient solutions, active ingredient emulsions and active ingredient suspensions.

In a preferred embodiment, the spots are applied in such a way that the spots applied in step (b) and the further steps (b) (according to step (e) of the method according to the invention) overlap with the spots applied in the previous step (step (b) or each step (b) of the further build-up steps according to step (e)). This embodiment thus results in a dosage form that can form a particularly stable arrangement in the sense of a brick arrangement of the applied spots, if the spots of a layer have a complete overlap with the preceding layer. On the other hand, by applying a layer of spots that partially overlap with the preceding layer, a particularly light arrangement with interstices is provided, whereby the solubility or degradation rate of the dosage form exposed to the surrounding environment can be controlled by creating a larger surface area of the dosage form exposed to the surrounding environment, in particular in the digestive tract of the subject taking the dosage form, for example a human patient.

As described above, in preferred embodiments of the invention, the build-up substance is present as a filament and the print head is adapted, in the case of 3D printing, to melt a quantity of the filament, preferably by the print head comprising heating means, as described above, to apply the array of spots of the build-up substance on the build-up platform in step (c) and the further array(s) of spots on the previous array of spots in step (d).

The solidification (at least to a semi-solid state) of the printed build-up substance as well as the bonding between the individual applied spots in step (c) can be performed in different ways. For example, in one embodiment, when a fusible material is used, the bonding between such spots may be performed by solidification after application to the support structure, which may be performed by various mechanisms such as simple cooling and/or chemically by known substances. In another embodiment, a suitable binder may be added to the build-up substance, e.g. a dispersion or a solution, which after application of the spot causes it to cure, wherein the curing by the binder may be effected, for example, by heat which may be supplied by a suitable heat source in the printing device, such as, for example, a light source, preferably a laser device. Curing by the binder may also be effected chemically by appropriate starter molecules and/or light of a suitable wavelength, the latter again being preferably emitted by means of a laser device. In a further embodiment, the build-up substance may contain one or more starting compounds, typically monomers, of one or more polymers, and after application of the spot(s), polymerization is initiated by suitable means such as, again, light, heat or other polymerization initiators, which cures and bonds the respective applied spot to adjacent spots.

Suitable carrier materials flowable at the printing temperature, in which the active ingredient(s) is/are present, are also in the case of the preferred spot printing process e.g. carriers generally usable for hot melt extrusion (HME), such as low-melting waxes and polymers. In addition to the low melting point carrier, the HME mixture or, more generally, the build-up mixture may contain other process agents and auxiliary substances such as binders, plasticizers, antioxidants, fragrances, sweeteners or the like. Suitable HME carriers and plasticizers are known to the skilled person and are disclosed, for example, in Crowley et al. (2007) Drug Development and Industrial Pharmacy, 33, 909-926 (carriers: pages 917 to 919, in particular Table 1; plasticizers: pages 917 and 920, in particular Table 2), with expressis verbis reference also being made to the aforementioned passages in the present description of the preferred spot printing process.

The method according to the invention is not limited to a complete de novo assembly of dosage forms or medical devices. The method can also be applied to objects already present on the assembly device. This includes, for example, dosage forms previously produced conventionally or in another manner, which are to be modified, for example, by the present method, or active substance-free objects (also referred to as placebo carriers) onto which volume increments containing active substances are printed in accordance with the invention. For example, drug-free films or other flat materials such as edible paper can be presented to provide drug-containing ODF (“orally degradable film” or “orally dissolvable film”) products. In other embodiments, provided plaster materials can be printed, for example, with volume increments according to the invention, which contain, for example, active substances that promote wound healing. In further embodiments, placebo carriers produced, for example, by a fused layer modelling process, can be printed by the process according to the invention with API-containing volume increments and subsequent printing of solvent-containing liquids, whereby the printed layers can alternate.

The printing process steps (step (3) and/or step(s) (6) of the method according to the invention) are preferably carried out computer-aided. Thus, a calculated three-dimensional image of the dosage form to be printed is typically generated, for example, with the aid of a common CAD program. The computer-generated representation of the object to be printed may also be obtained by scanning an existing dosage form. In the present method, the computer-generated model image is then subdivided into the desired volume increments (voxels), spots or desired filament elements, which are in principle freely selectable, whereby the smaller the volume increments, the greater the resolution of the real object. Each individual volume increment can, for example, be assigned an active ingredient, carrier or base substance or carrier or base compositions and/or further auxiliaries such as coloring substances and other materials that may be required, as well as their quantity (concentration in the volume increment) and finally printed. Suitable printing devices for the preferred jetting process are described, for example, in US 2017/0368755 A1 and U.S. Pat. No. 6,070, 107.

In a particularly preferred embodiment, the method according to the invention further comprises, in step (3) and/or step(s) (6), applying at least one colored substance by means of 2D and/or 3D printing, preferably also by printing corresponding voxels, preferably small-volume voxels, according to the preferred jetting process, and/or by another process, such as, for example, by two-dimensional printing as in inkjet printing, on the dosage form or on at least a portion thereof in such a way that the applied substance forms at least one information structure visible on the dosage form. In this regard, the colored substance(s) may be applied separately from the active ingredient-containing volume increment(s). Preferably, the colored substance(s) can be applied together with volume increments containing active pharmaceutical ingredients. In one embodiment, one substance at a time can thus mark the area(s) or section(s) to which the respective active ingredient has been applied. This embodiment can therefore convey, by color coding, information about the active ingredients present in the object and their distribution in the complete object. In a further development of this embodiment of the invention, different amounts or concentrations of the respective active ingredient can be deposited in the active ingredient-containing sections, which in turn are reflected by the concentration of the respective color substance. Of course, different color substances can also be mixed, e.g. in one voxel, so that the entire visible spectrum can generally be used by appropriate selection of the mixture(s).

According to the invention, the term “color substance” also includes substances that luminesce, in particular fluoresce.

The information structure caused by the color substance(s) can depict a variety of information, whereby several different information structures can also be used by means of different color substances, which are essentially freely selectable and combinable. In particular, it is provided according to the invention that the at least one information structure encodes information on the type of active substance(s) printed in the object and/or on the amount(s) of active substance(s) present in the dosage form and/or on the time or period of administration provided for a dosage form and/or on the date of administration provided for the dosage form and/or on patient-related data (such as, for example, name, age, sex, medication and disease(s)) and/or the health and care insurance provider and/or the attending physician and/or the pharmaceutical company providing the pharmaceutical form and/or the medical institution providing the pharmaceutical form (or a medical device) are encoded.

The information structure can be selected from a wide range of application possibilities. For example, the substance or substances can be printed in the form of QR codes, letters and/or numbers. Of course, a wide variety of patterns such as lines, grids, dots, two-dimensional patterns, etc. can also be printed, whereby the preferred embodiment of voxel printing basically offers the most diverse possibilities. The printed image of the code can thus contain the active ingredient and simultaneously encodes the desired data, as described above, about the patient, doctor, pharmacist and/or medical or pharmaceutical professional.

It is apparent to the skilled person that, depending on the specific additive manufacturing process chosen, the coloring substance(s), if present, may be present together with the active pharmaceutical ingredient(s) in the respective printed base composition. In the present method, it is preferred that the coloring substance (or several thereof) is preferably present together with the active ingredient(s) in the build-up substance provided for a given volume increment.

As outlined above, the dosage form may contain a wide variety of information structures, preferably those mentioned in the method described above.

Objects containing active ingredients that can be printed using the process according to the invention are in particular semi-solid or solid pharmaceutical dosage forms, such as tablets, capsules, implants, patches, suppositories or thin films. Tablets producible by the method of the invention are diverse and include oblong tablets, lozenges, implant tablets, multiple application tablets, dispersible tablets, sustained release tablets, vaginal tablets and suppositories, ophthalmic tablets, coated tablets, matrix tablets, chewable tablets, film-coated tablets, modified-release tablets, lacquer tablets, and enteric-coated tablets and drug-abuse deterrent tablets.

Further, particularly suitable objects which may be considered in the context of the present invention as a dosage form of active pharmaceutical ingredients are medical devices such as topical dosage forms containing active ingredients, contact lenses, plasters, which preferably release the active ingredient(s) for local application.

In step (4) of the method according to the invention, the individual and disease-related parameters of the patient are again analyzed, whereby, after the patient has thus been provided with the dosage form(s) printed by step (3), the patient is now under therapy with this dosage form (or the dosage forms) printed in step (3), these parameters are therefore influenced by the dosage form(s) printed in step (3).

In step (5) of the method according to the invention, at least one administration-relevant parameter of the active substance or the active substances is adapted on the basis of the analysis of the parameters in step (4), and finally, in step (6), a new dosage form is created in 2D and/or 3D printing using the at least one adapted parameter.

As set out in the optional step (7) of the method, the steps of analysis (4), adjustment of the administration-relevant parameter(s) of the active substance(s) (5) and printing of the dosage form(s) (6) can be repeated on the basis of the adjusted parameter(s), which is preferred according to the invention, in particular in order to continuously adapt the dosage form(s) to the possibly changing individual and disease-related parameters.

It is apparent to one skilled in the art that printing processes used in step (3) and the step(s) (6) of the process according to the invention may be the same or different. Thus, in certain embodiments, an extrusion process (e.g. the preferred process described in more detail above or another 3D/2D extrusion printing process known per se) may be used in step (3) and a jetting process (e.g. the process of a preferred process described in more detail above) or a spot printing process (e.g. the process described in more detail above), or vice versa, may be used in one or further step(s) (6), whereby all combination possibilities can be selected, in particular also depending on the administration-relevant parameter to be adjusted.

Following on from the example of the immunosuppressants, an analysis of the individual and disease-related parameters is therefore first used to select at least one administration-relevant parameter (such as, for example, the dose of one or more of the immunosuppressants). (e.g. the dose of one or more of the immunosuppressants), a (first) dosage form is printed (which, according to the invention, may contain one or more of the immunosuppressants mentioned by way of example) either on the basis of the initial parameter (if a new medication is given (step (1a)) or a different active ingredient has been selected (step (1b)) or on the basis of the changed parameter in comparison with the previous medication. With reference to the immunosuppressants mentioned above by way of example, it is possible according to the invention to print respective three different dosage forms (one dosage form for each of the three immunosuppressant classes mentioned) or to combine two or all three immunosuppressants in one dosage form, in which case appropriate release properties and/or suitable release layers, if required, are usually included in a multiple dosage form in the printing process, as described in detail in the context of the preferred printing processes. As already explained above using the example of immunosuppressants and corticoids, the process according to the invention can also be used for patient-optimized up-titration and/or down-titration of one or more active ingredients.

After analysis of the individual and disease-related parameters of the patient under therapy with the dosage form(s) initially printed according to the invention, one or more administration-relevant parameters are in turn adapted on the basis of the analysis and one or more dosage forms of the immunosuppressants are again printed in order to optimize the immunosuppression therapy and to adapt it to the current requirements, so that in the long term the success of the therapy can be improved, i.e. in the case of the immunosuppressants in the context of preventing transplant rejection, the survival of the transplant can be prolonged.

In addition to steps (3) and (6) described above, which are preferably computer-assisted, the other steps of the method according to the invention are also preferably computer-assisted.

According to the invention, the analysis in step (1) and in step(s) (4) is computer-assisted, whereby the respective influencing parameters (i.e. individual and/or disease-related parameters) for a dose adjustment can be determined by the physician or the pharmacist. Possible influencing parameters are already mentioned above and include, for example, age, weight, body surface area, height, liver status, kidney status, metabolic influences, general condition of the patient, gender, previous illnesses, social status and adherence to therapy, drug safety, adherence, active ingredient(s), active ingredient concentration(s), incompatibilities, allergies, interactions and intake compliance(s).

The algorithm is calculated using statistical analysis and questionnaires, which are calculated using mathematical models to calculate the optimal dose for a wide range of patients treated with a specific agent. The formulation of the personalized medicines can also influence the evaluation and the resulting dose adjustment.

Likewise, according to the invention, the determination of at least one administration-relevant parameter in step (2) and the adjustment of the at least one administration-relevant parameter in step(s) (5) is computer-assisted. For this purpose, questionnaires and therapy success factors are usually determined, which then correspond to the variable parameters and vital data of the patient. These form the basis for the subsequent evaluation via an Al learning system.

This also preferably applies to the conversion of the at least one administration-relevant parameter into one or more printing parameters for 3D and/or 2D printing.

1. A method for producing patient-optimized pharmaceutical dosage forms comprising the steps of:
(1) analyzing of individual and/or disease-related data of a patient suffering from a disease state, wherein, optionally, the patient may be exposed to an active ingredient for the treatment of the patient's disease state;
(1a) where appropriate, selecting one or more active ingredients to treat the patient's disease state, if the patient has not already been exposed to an active ingredient to treat the disease state; or
(1b) where appropriate, selecting one or more other active ingredients for the treatment of the patient's disease state, if the previous active ingredient(s) has disadvantages compared to the other active ingredient based on the analysis of step (1) for the treatment of the patient's disease state;
(2) determining at least one administration-relevant parameter of the previous active ingredient or of the active ingredient(s) selected according to step (1a) or (1b) for a pharmaceutical form from the analysis of the data according to step (1), where appropriate taking into account potential side effects and/or influences of further active ingredients for the treatment of the same or another disease of the patient to which the patient is exposed;
(3) printing a first dosage form containing the active ingredient(s) according to the at least one administration-relevant parameter determined in step (2) by means of 3D and/or 2D printing, wherein the at least one administration-relevant parameter is converted into one or more corresponding printing parameters for the 3D and/or the 2D printing;
(4) analyzing individual and disease-related data of the patient under administration of the first dosage form;
(5) adjusting the at least one administration-relevant parameter of the active substance or substances for a dosage form according to the analysis of the data according to step (4);
(6) printing a further dosage form containing the active ingredient(s) according to the at least one administration-relevant parameter adjusted in step (5) by means of 3D and/or 2D printing; and, optionally
(7) repeating steps (4) to (6).

2. The method of claim 1 wherein the administration-relevant parameter(s) is/are selected from the group consisting of amount of active ingredient(s) per unit dose of the dosage form, release kinetics of the active ingredient(s) from the dosage form at the site of administration and/or along the route of the dosage form in the patient, concentration of the active ingredient(s) in the dosage form, distribution of the concentration of the active ingredient(s) in the dosage form, size of the dosage form, geometric shape of the dosage form, coating parameters of the dosage form, surface structure of the dosage form, inner structure of the dosage form, distribution of the active ingredient(s)) in the dosage form and combinations of two or more thereof.

3. The method of claim 1 wherein the individual parameters of the patient are selected from the group consisting of age, gender, developmental state, genetic predispositions, height, weight, body surface area, body mass index, general physical condition, drug consumption, eating and drinking habits, sleeping habits, physical activity, and combinations of two or more thereof.

4. The method according to claim 1 wherein the disease-related data are selected from the group consisting of blood pressure, heart rate, ECG findings, EEG findings, sonographic findings, CT findings, MRI findings, biopsy findings of diseased tissue, blood count, electrolyte blood levels, blood liver levels, nephrological blood and urine levels, blood lipid levels, blood glucose levels, vitamin metabolism data, metabolic interactions, medication schedule, side effect profiles, urine status, virological findings, bacteriological findings, fungal findings, parasitic findings, stage of disease, course of disease and combinations of two or more thereof.

5. The method according to claim 1 wherein the disease state is selected from the group consisting of diseases of internal organs, rheumatological diseases, oncological diseases, cardiovascular diseases, neurological diseases and hematological diseases.

6. The method of claim 5 wherein the disease of internal organs is selected from the group consisting of kidney, liver and pancreas transplantations.

7. The method of claim 6 wherein the active ingredient(s) is/are selected from the group consisting of glucocorticoids, calcineurin inhibitors and inhibitors of inosine monophosphate dehydrogenase.

8. The method of claim 5 wherein the disease state is a rheumatological disease and the active ingredient(s) is/are selected from the group consisting of glucocorticoids, calcineurin inhibitors, inhibitors of inosine monophosphate dehydrogenase and inhibitors of tyrosine kinase.

9. The method of claim 5 wherein the disease state is an oncological disease and the active ingredient(s) is/are selected form inhibitors of tyrosine kinase.

10. The method of claim 5 wherein the neurological diseases is Morbus Parkinson and the active ingredient(s) is/are selected from dopamine antagonists.

11. The method wherein the hematological disease is an anemia.

12. The method of claim 11 wherein the active ingredient(s) is/are selected from the group consisting of iron preparations, vitamin B12 and folic acid.

13. The method of claim 5 wherein the cardiovascular diseases are selected from the group consisting of high blood pressure, stroke, ventricular fibrillation and risk for heart attack.

14. The method of claim 13 wherein the active ingredient(s) is/are selected from the group consisting of antihypertensive drugs and anticoagulants.

15. The method of claim 14 wherein the anticoagulants are selected from the group consisting vitamin K antagonists, thrombin inhibitors and factor Xa inhibitors.

16. The method of claim 14 wherein the antihypertensive drugs are selected from the group consisting of calcium antagonists, betablockers, ACE inhibitors, diuretics and AT1 inhibitors.

17. The method according to claim 1 the analysis in step (1) and in step(s) (4) is carried out computer-aided.

18. The method according to claim 1 wherein the determination of the at least one administration-relevant parameter is in step (2) and the adjustment of the at least one administration-relevant parament in step(s) (5) is carried out computer-aided.

19. The method according to claim 1 wherein the conversion of the at least one administration-relevant parameter into one or more printing parameters for the 3D and/or 2D print is carried out computer-aided.

20. A method for producing patient-optimized pharmaceutical dosage forms comprising:
printing a pharmaceutical dosage form containing one or more active ingredient(s) selected from the group consisting of glucocorticoids, calcineurin inhibitors, inhibitors of inosine monophosphate dehydrogenase, inhibitors of tyrosine kinase, dopamine antagonists, iron preparations, vitamin B12, folic acid, calcium antagonists, betablockers, ACE inhibitors, diuretics, AT1 inhibitors, vitamin K antagonists, thrombin inhibitors and factor Xa inhibitors;
wherein the pharmaceutical dosage form has a size, shape, inner structure and outer structure, and contains an amount of active ingredient(s) selected for a specific patient; and
wherein the pharmaceutical dosage form is printed using filament fusion fabrication (FFF), fused layer modelling (FLM), voxel printing, binder jetting, or spot printing.
